(19) United States
(12) Patent Application Publication
(10) Pub. No.: US 2020/0087690 A1
(43) Pub. Date: Mar. 19, 2020

(54) A METHOD OF INHIBITING ISOMERIZATION OF A REDUCING SACCHARIDE UPON THERMAL TREATMENT

(71) Applicant: Jennewein Biotechnologie GmbH, Rheinbreitbach (DE)

(72) Inventor: Dirk WARTENBERG, Bonn (DE)

(21) Appl. No.: 16/493,996

(22) PCT Filed: Mar. 16, 2018
(86) PCT No.: PCT/EP2018/056727

§ 371 Date: Sep. 13, 2019

(30) Foreign Application Priority Data
Mar. 17, 2017 (EP) 17161671.7

(51) Int. Cl. C12P 19/12; C13K 5/00

(52) U.S. Cl. CPC C12P 19/12; C13K 5/00

(57) ABSTRACT

Disclosed is a method of inhibiting isomerization of a reducing saccharide in an aqueous solution containing said reducing saccharide upon thermal treatment of said aqueous saccharide solution by acidifying the aqueous saccharide solution prior to its thermal treatment, and the use of the thermally treated aqueous solution containing said reducing saccharide for producing a biological product.

The present invention relates to methods involving a thermal treatment of an aqueous solution containing at least one reducing saccharide. More specifically, the invention relates to methods of preventing isomerization of the reducing saccharide in an aqueous solution upon a thermal treatment of aqueous solution containing the reducing saccharide.

### BACKGROUND

Maintaining a sterile environment is often a prerequisite for cultivating cells in biotechnological production processes. However, these processes are often prone to foreign growth by adventitious bacteria, fungi or viruses. Being contaminated with adventitious microorganisms has a severe impact on the manufacturing process as it impairs productivity (decreased production capacity due to a degradation or modification of the raw materials, the desired product or the desired biomass, and extended shut down periods of the bioreactor for removal of the contaminant), product quality and product safety (through contaminations of the final product due to the foreign growth itself and/or due to metabolites produced by the undesired microorganisms).

Preventing foreign growth on a biological product as well as in the process of its production is challenging due to the ubiquitous nature of microorganisms and multiple points of microbial entry into the production process. Various methods have been developed for sterilizing equipment (e.g. the interior of a fermenter), growth media as well as any supplements to the growth media that have to be used in a biotechnological production process.

Several methods are known for sterilizing compounds or compositions and are employed to eliminate adventitious microorganisms from supplies to be used in biotechnological production processes. Such methods include gas sterilization (e.g. with ozone or ethylene oxide), radiation sterilization (e.g. ultra violet radiation or gamma radiation), bright light/pulsed light sterilization as well as sterile filtration of liquids and solutions, in particular of liquids and solutions that are heat-sensitive or contain at least one heat-sensitive compound.

Heat treatment of any heat-tolerant supplies is known to be the most reliable and effective sterilization method and wet heat is most widely used for achieving heat sterilization. Wet heat (steam) sterilization means exposing the components to be sterilized to pressurized steam for some time. Typical wet heat sterilization protocols that are used for sterilizing equipment and supplies subject them to high-pressure saturated steam at 115° C. to 140° C. for around 60 to 3 minutes. These sterilization protocols may vary depending on the bioburden and nature of the raw material, solution or surface to be sterilized.

Fundamentally, damaging a component by an inappropriate sterilization method may affect quality, safety or productivity of a biotechnological production process, thus, has to be avoided. Especially fluids may undergo various chemical reactions due to sterilization methods using heat, irradiation or chemicals.

The complexity of such chemical reactions is exemplified by heat-treating milk which is typically employed to reduce the microbial load or to inactivate enzymes in the milk, and hence extend the shelf-life of milk. Maillard reactions are known to occur which affect the color and taste of the milk upon its conventional sterilization at 121.1° C. for 20 minutes. Furthermore, the denaturation or inactivation of proteins or vitamins as well as reactions of aldo sugars with amino acids or amino group containing substances is known to occur due to the heat treatment of milk. Besides, it is also well known that lactose (4-O-β-D-galactopyranosyl-D-glucopyranose, CAS-number: 63-42-3), a substantial constituent of milk, isomerizes to lactulose (4-O-β-D-galactopyranosyl-D-fructofuranose, CAS-number: 4618-18-2) due to heat treatment and gets further degraded to glucose and galactose as well as to further degradation products such as acids. This type of isomerization is favored by basic pH and is also known as the Lobry de Bruyn-Alberda van Ekenstein transformation.

In attempts to limit the undesired effects of heat to the quality and composition of milk, alternative methods of sterilization are used such as “ultra-high-temperature treatment”, wherein the milk is exposed to 140° C. for a couple of seconds to few minutes, or “low temperature pasteurization” wherein milk is heated to a temperature of up to 74° C. Nevertheless, the undesired heat-mediated reactions also occur during these alternative sterilization methods, although to a lesser extent.

This could be further improved by adjusting the pH values of raw skim milk, having an initial pH of 6.70, to values of between 6.59 and 6.72 prior to its sterilization at 120° C. for 10 minutes. This led to a reduced lactulose formation by 28% at pH 6.59 and a 9% increased lactulose formation at pH 6.72 as compared to lactulose formation in the original milk.

In the biotechnological production of human milk oligosaccharides such as 2′-fucosyllactose, 3-fucosyllactose, lacto-N-tetraose, lacto-N-neotetraose, 3′-sialyllactose or 6′-sialyllactose, lactose is typically used as initial acceptor molecule for further glycosylation steps leading to the desired HMO to be produced. For preventing foreign growth in the fermentation broth, the lactose being supplied has to be sterilized. However, the presence of lactulose within the fermentation broth has to be avoided since it is a known laxative that should not be present in infant formula or any other nutritional product being supplemented with said HMOs. Furthermore, lactulose might be used by the HMO producing bacteria as an alternative acceptor molecule, thus, leading to oligosaccharides which are not present in nature.

As an alternative to heat treatment, it is possible to sterilize a lactose solution by filtration. Typically, solutions containing between 1 mM and 1 M lactose are sterile filtered. However, sterilizing large amounts of a solution by filtration as necessary for industrial scale production is costly, time consuming and less reliable as compared to heat sterilization. Therefore, a reliable, more convenient way of sterilizing lactose with no or only conversion of minute amounts of lactose to lactulose is needed.

The object has been achieved by a method wherein an acidic pH of a lactose solution is adjusted prior to and/or in the course of exposing the lactose solution to heat, notwithstanding that the principle of acidifying a sugar solution prior to its heat treatment can be applied to other saccharides than lactose as well.

### SUMMARY

In a first aspect, the present invention provides a method of inhibiting isomerization of a reducing saccharide in an aqueous solution containing said reducing saccharide upon thermal treatment of said aqueous solution by acidifying the aqueous solution prior to and/or in the course of its thermal treatment.

In a second aspect, the present invention provides a thermally treated aqueous solution containing at least one reducing saccharide.

In a third aspect, the present invention provides the use of a thermally treated aqueous solution containing at least one reducing saccharide in a biotechnological production of a biological product.

In a fourth aspect, the invention provides methods of producing a biological product, wherein a thermally treated aqueous solution containing at least one reducing saccharide is employed.

In a fifth aspect, the invention provides a biological product produced by biotechnological production utilizing a thermally treated aqueous solution containing at least one reducing saccharide.

In a further aspect, the invention provides the use of the biological product produced by biotechnological production utilizing a thermally treated aqueous solution containing at least one reducing saccharide for manufacturing a formulation.

In another further aspect, the invention provides a formulation comprising a biological product that has been produced by a biotechnological production utilizing a thermally treated aqueous solution containing at least one reducing saccharide.

### BRIEF DESCRIPTION OF THE DRAWINGS

FIG. 1 displays chromatograms of an aqueous solution containing lactose (A) prior to heat sterilization and (B) after heat sterilization. The aqueous solution was not acidified prior to its heat sterilization. FIG. 1C shows a chromatogram of various specific saccharides used as standards.

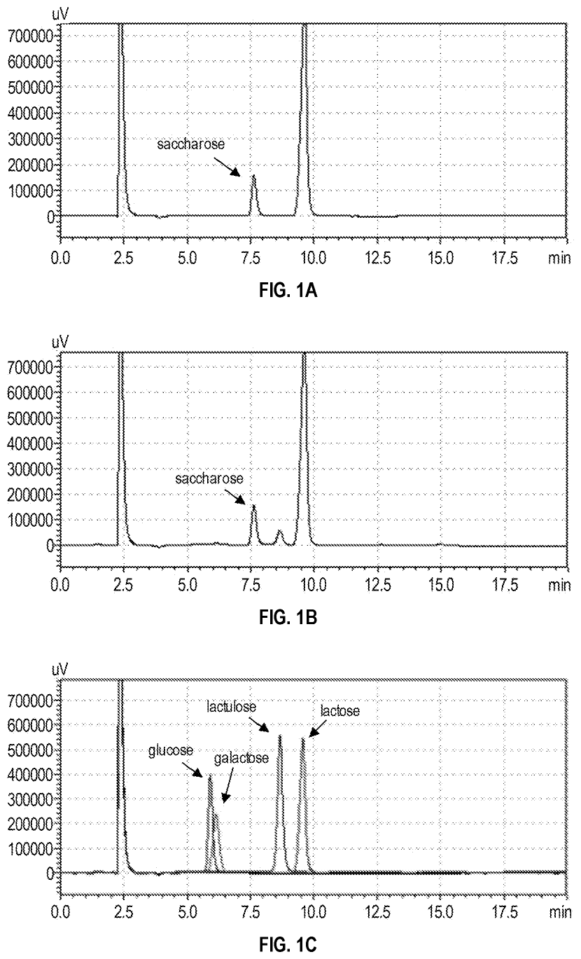

FIG. 2 displays chromatograms of an aqueous solution containing lactose (A) prior to heat sterilization and (B) after heat sterilization. The aqueous solution was acidified prior to its heat sterilization by adding sulfuric acid to the aqueous solution containing lactose. FIG. 2C shows a chromatogram of various specific saccharides used as standards.

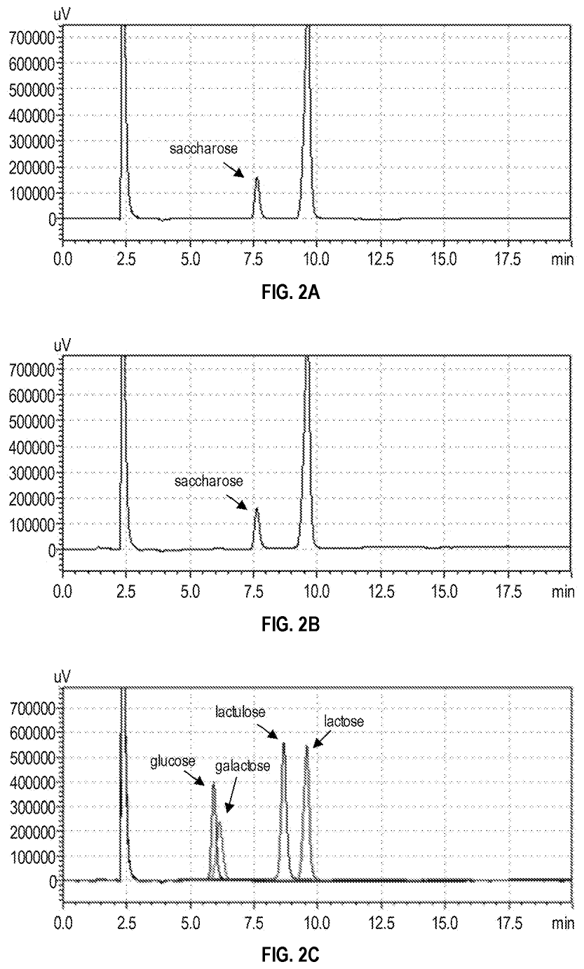

### DETAILED DESCRIPTION

According to the first aspect, provided is a method of inhibiting isomerization of a reducing saccharide in an aqueous solution containing said reducing saccharide (aqueous saccharide solution) upon thermal treatment of said aqueous saccharide solution, the method comprising the step of acidifying the aqueous saccharide solution prior to and/or in the course of its thermal treatment.

The term “reducing saccharide” as used herein refers to any sugar or saccharide that is capable of acting as a reducing agent because it has a free aldehyde group. The reducing saccharide comprises monosaccharides, disaccharides and oligosaccharides. All monosaccharides are reducing sugars, they can be classified into aldoses, which have an aldehyde group, and the ketoses, which have a ketone group. Ketoses must first tautomerize to aldoses before they can act as reducing sugars. Disaccharides are formed from two monosaccharide residues and oligosaccharides are formed from three to seven monosaccharide residues. Disaccharides and oligosaccharides can be classified as either reducing or nonreducing. Reducing disaccharides like lactose and maltose have only one of their two anomeric carbons involved in the glycosidic bond, meaning that they can convert to an open-chain form with an aldehyde group.

In an additional and/or alternative embodiment, the reducing saccharide is selected from the group consisting of aldoses, disaccharides and oligosaccharides.

The term “aldose” as used herein refers to monosaccharides that contain only one aldehyde group per molecule. Examples of aldoses are D-(+)-glyceraldehyde, D-(−)-erythrose, D-(−)-threose, D-(−)-ribose, D-(−)-arabinose, D-(+)-xylose, D-(−)-lyxose, D-(+)-allose, D-(+)-altrose, D-(+)-glucose, D-(+)-mannose, D-(−)-gulose, D-(−)-idose, D-(+)-galactose, and D-(+)-talose.

In an additional and/or alternative embodiment, the disaccharide is selected from the group consisting of lactose, maltose, trehalose, cellobiose, chitobiose, kojibiose, nigerose, isomaltose, sophorose, laminaribiose, gentibiose, turanose, matulose, palatinose, gentibiose, mannobiose, melibiose, melibiulose, rutinose, rutinulose and xylobiose.

The term “oligosaccharide” as used herein refers to saccharides consisting of 3, 4, 5, 6, or 7 monosaccharide residues, and thus comprises trisaccharides, tetrasaccharides, pentasaccharides, hexasaccharides and heptasaccharides.

For obtaining the aqueous saccharide solution, an amount of at least one reducing to saccharide is dissolved in a supply of water. Said water may be selected from the group consisting of distilled water, double distilled water, deionized water, groundwater, river water, seawater, tap water, municipal water and saline-containing water. The term “saline-containing water” as used herein refers to an aqueous solution of one oe more salts. In an additional and/or alternative embodiment, the aqueous saccharide solution does not comprise one or more selected from the group consisting of proteins, polypeptides, nucleic acids (such as DNA and/or RNA) and lipids (such as fatty acids, mono-, di- and/or triglycerols).

In an embodiment of the method, the aqueous saccharide solution is acidified to a pH having a value of between about 1 to about 6, preferably to a pH having a value of between about 2 to about 5, and more preferably to a pH having a value of between about 3 and about 4. A pH of the aqueous saccharide solution having a value of between about 3 to about 5 was found to be of particular advantage, because isomerization of the reducing saccharide is inhibited or even prevented while formation of degradation products of said reducing saccharide is negligible.

In an additional and/or alternative embodiment, the aqueous saccharide solution is acidified by adding an acid to the aqueous saccharide solution. The acid can be any acid which does not lead to an undesired chemical reaction with the reducing saccharide. An example of such an undesired chemical reaction is the formation of mucic acid if nitric acid is added to an aqueous lactose solution.

The acid can be selected from the group of organic acids and inorganic acids, with the provision that the inorganic acid is not nitric acid (or nitrous acid) if the reducing saccharide is galactose or a galactose-containing saccharide, as nitric acid oxidation of galactose or galactose-containing compounds such as lactose leads to mucic acid.

In an additional and/or alternative embodiment, the at least one acid for acidifying the aqueous saccharide solution is an inorganic acid or mineral acid. The inorganic acid is a suitable inorganic acid which—at the amount to be added to the aqueous saccharide solutions—does not inadvertently react with the saccharide. For example, adding nitric acid to an aqueous lactose solution may lead to mucic acid. The inorganic acid is preferably selected from the group consisting of hydrochloric acid, sulfuric acid, sulfurous acid, phosphoric acid, boric acid, hydrofluoric acid, hydrobromic acid, perchloric acid, hydroiodic acid, and carbonic acid.

In the embodiment, wherein the inorganic acid is carbonic acid, the aqueous saccharide solution can be acidified in that the aqueous saccharide solution is gassed with carbon dioxide in a pressurized container.

In an additional and/or alternative embodiment, the at least one acid for acidifying the aqueous saccharide solution is an organic acid. The organic acid may be selected from the group consisting of monocarboxylic acids, dicarboxylic acids, and tricarboxylic acids.

In an additional and/or alternative embodiment, the monocarboxylic acid is selected from, but not limited to, the group consisting of carbonic acid, formic acid (methanoic acid), acetic acid (ethanoic acid), proprionic acid (propanoic acid), butyric acid (butanoic acid), and valeric acid (pentanoic acid).

In an additional and/or alternative embodiment, the dicarboxylic acid is selected from the group consisting of oxalic acid, malonic acid, succinic acid, glutaric acid, adipic acid, maleic acid, malic acid, fumaric acid, glutaconic acid, muconic acid, and citraconic acid.

In an additional and/or alternative embodiment, the tricarboxylic acid is selected from the group consisting of citric acid, isocitric acid, and aconitic acid.

Adjusting the pH of the saccharide solution to an acidic value permits a thermal treatment of the reducing saccharide in the aqueous solution without or at least with a reduced isomerization of the reducing saccharide.

The term “thermal treatment” as used herein encompasses warming or heating and/or keeping the aqueous saccharide solution at an elevated temperature, i.e. a temperature above room temperature (21° C.). Thermal treatment of the aqueous saccharide solution comprises heating the aqueous saccharide solution after acidification and/or while acidifying to a temperature of about 30° C., about 40° C., about 50° C., about 60° C., about 70° C. or even about 80° C., and also includes keeping the aqueous saccharide solution at such a temperature—optionally after it has been heated to even higher temperatures—for an extended period of time, i.e. for several hours or even days, such as—for example, but not limited thereto—for about 20 hours, about 30 hours, about 40 hours, about 50 hours, about 60 hours, about 72 hours or even longer.

Heating and/or keeping an aqueous solution containing a reducing saccharide at such elevated temperatures without isomerization or with significantly reduced isomerization of the reducing saccharide provides multiple advantages such as—for example—the option of dissolving a higher amount of the saccharide in a given amount of water thereby increasing the saccharide concentration in the aqueous solution and reducing the volume of an aqueous saccharide solution to be supplied to a batch for obtaining a desired final saccharide concentration. Additionally and/or alternatively, the viscosity of the aqueous saccharide solution can be decreased by heating/keeping the aqueous saccharide solution at an elevated temperature, thereby facilitating handling and/or pumping of the aqueous saccharide solution, e.g. through a membrane filter.

The term “thermal treatment” as used herein also encompasses heating and/or keeping the aqueous saccharide solution for some time at an elevated temperature which is suitable for sterilizing the aqueous saccharide solution. Hence, “thermal treatment” also comprises heating the aqueous saccharide solution to a temperature in the range of about, but not limited to, 115° C. to 150° C. and keeping the temperature for up to about 60 minutes.

In an embodiment of the thermal treatment for sterilizing the aqueous saccharide solution, the aqueous saccharide solution is sterilized by autoclaving. Autoclaving is one of the most important methods of germ destruction wherein saturated, superheated steam is utilized. The condensation of steam on the object to be sterilized releases energy which causes irreversible damage to the microorganisms.

To this end, the interior of the autoclave is vented during the intial rise time. In doing so, the atmospheric air is displaced from the interior and replaced by saturated, superheated steam. Venting takes place using a flow process or through fractioned venting; once venting is complete, the vent valve is closed. This marks the start of the compensation time. After this period, every point of the item to be sterilised reaches the required temperature due to the effect of the saturated steam. After this, the actual sterilisation phase begins. The duration of sterilisation is dependent on both germ loading and sterilisation temperature. Autoclaving at 121.1° C. (250° F.) for 15 minutes to 30 minutes is seen as standard. Vegetative forms, encompassing procaryotic and eucaryotic organisms as well as viruses/bacteriophages, can usually be inactivated within a few minutes at temperatures of 65° C.-100° C. whereas survival forms such as spores may have to be treated at temperatures up to 140° C. Prions require at least 30 minutes at 132° C. to 134° C. and 3 bar pressure in order to be inactivated or destroyed. The subsequent cool-down phase, and thus the end of the autoclave cycle, starts after the sterilisation time.

In an alternative embodiment of the thermal treatment for sterilizing the aqueous saccharide solution, the aqueous saccharide solution is sterilized by a process called “ultra-high-temperature treatment”, a continuous sterilization method, which comprises heating the aqueous to a temperature of 130° C.-150° C. with 140° C. as a main point. The corresponding holding time may vary from 8 to 40 seconds, occasionally to up to 5 minutes, depending on the properties of the solution to be sterilized.

In yet another embodiment of the thermal treatment for sterilizing the aqueous saccharide solution, the aqueous saccharide solution is subjected to a high temperature/short time (HTST) pasteurization, in which the solution is heated to a temperature of between 71.5° C. to 74° C., preferably to 72° C. for about 15 seconds to about 30 seconds, and is moved in a controlled, continuous flow while subjected to said thermal treatment.

In yet another embodiment of the thermal treatment for sterilizing the aqueous saccharide solution, the aqueous saccharide solution is subjected to “flash pasteurization”, wherein the aqueous saccharide solution is subjected to 71.7° C. for 15 seconds.

Acidifying an aqueous solution of a reducing saccharide prior to subjecting the aqueous saccharide solution to any of these thermal treatments and/or in the course of its thermal treatment for sterilizing the aqueous saccharide solution inhibits or even prevents isomerization of the reducing saccharide in the aqueous solution upon its heat treatment.

Thus, according to the second aspect, a thermally treated aqueous solution containing at least one reducing saccharide is provided which is obtained by the method according to the first aspect, i.e. by a method of inhibiting isomerization of said reducing saccharide in an aqueous solution of said reducing saccharide, including the acidification of the aqueous saccharide solution prior to and/or in the course of a thermal treatment of said aqueous saccharide solution.

The thermally treated aqueous solution containing at least one reducing saccharide which is obtained by the acidification of the aqueous saccharide solution prior to and/or in the course of its thermal treatment and which contains no or at least less amounts of undesired isomerization products of said at least one reducing saccharide as compared to a similar aqueous solution of the same reducing saccharide which was not acidified prior to an identical thermal treatment.

In an embodiment of the second aspect, the aqueous solution containing a reducing saccharide is a sterile aqueous solution. The sterile aqueous solution containing a reducing saccharide is obtained by the method of inhibiting isomerization of said reducing saccharide as described herein before, including the thermal treatment of said aqueous saccharide solution for sterilizing said aqueous saccharide solution. Thus, aqueous solution has been sterilized by the thermal treatment.

In an additional and/or alternative embodiment, the aqueous solution containing a reducing saccharide has an elevated temperature, i.e. a temperature of about 30° C., about 40° C., about 50° C., about 60° C., about 70° C. or even about 80° C., and contains the reducing saccharide in an amount that is higher than the amount of said reducing saccharide that can be dissolved in water at room temperature. For example, an aqueous solution containing lactose in a concentration of 10 mM, preferably in a concentration of 100 mM, more preferably in a concentration of 0.66 M, most preferably in a concentration of of 1 M, is provided if the temperature of the acidified aqueous lactose solution is maintained at about 40° C. to about 60° C.

The aqueous saccharide solution containing at least one reducing saccharide, such as—for example—lactose, in an amount that is higher than the amount of the saccharide that can be dissolved in water at room temperature may be a sterile aqueous lactose solution that has been sterilized by means of a thermal treatment for sterilization as described herein before and allowed to cool down to the desired elevated temperature at which the sterile aqueous saccharide solution is kept.

According to the third aspect, the invention provides the use of a thermally treated aqueous solution containing a reducing saccharide as described herein before in a biotechnological production of a biological product. The production of the biological product

In an embodiment, the use of the thermally treated aqueous solution containing a reducing saccharide in a biotechnological production of a biological product comprises the use in a biocatalytic production process.

The term “biocatalytic production process” as used herein is understood to refer to a process for producing a biological product wherein one or more purified or isolated enzymes are contacted with one or more educts in an in vitro reaction to convert the one or more educts to the desired biological product.

In an alternative embodiment, the use of the thermally treated aqueous solution containing a reducing saccharide comprises the use in a fermentative production process. The term “fermentative production process” as used herein refers to a process wherein microorganisms are grown in a medium or broth with the aim of producing a biological product or specialty product that is synthesized by the microorganisms.

Using a thermally treated aqueous solution containing at least one reducing saccharide, wherein the aqueous saccharide solution has been acidified as described herein before prior to and/or in the course of the thermal treatment of the aqueous saccharide solution is advantageous, among others, in that no or less undesired isomerization products of the reducing saccharide are present in the aqueous saccharide solution, and that no or less undesired isomerization products are supplied to the biotechnological production process as compared to a similar aqueous saccharide solution that was not acidified prior to its thermal treatment. Thus, there is no necessity remove the undesired isomerization products from the heat treated aqueous saccharide solution before it can be used in a biotechnological production process.

According to the fourth aspect, the invention provides methods for biotechnological production of a biological product.

In an embodiment of the method for biotechnological production of a biological product, the method is a biocatalytic production process. The method comprises the steps of
providing at least one purified enzyme;
contacting the at least one purified enzyme with one or more educts in the presence of the thermally treated aqueous saccharide solution, that was acidified as described herein before prior to and/or in the course of the thermal treatment, to reaction to convert the one or more educts to the desired biological product; and
optionally, purifying the biological product.

In an embodiment, the at least one reducing saccharide of the aqueous saccharide solution represents an educt of the biocatalytic production process.

In an alternative embodiment of the biotechnological production process, the method is a fermentative production process.

“Fermentation” or “fermentative” refers to the bulk growth of microorganisms on or in a growth medium (fermentation broth) with the goal of producing a specific chemical product, the “biological product”. To this end, cells of one or a limited number of strains of microorganisms are grown in a bioreactor (fermenter) under optimum conditions for the microorganisms to perform the desired production with limited production of undesired impurities. The environmental conditions inside the bioreactor, such as temperature, nutrient concentrations, pH, and dissolved gases (especially oxygen for aerobic fermentations) affect the growth and productivity of the organisms, and are therefore monitored, controlled and adjusted if necessary.

Thus, the method of fermentative production of a biological product comprises the steps of:
providing a cell that is capable of producing the biological product;
cultivating the at least one cell in a fermentation broth containing and/or being supplemented with the thermally treated aqueous solution containing the at least one reducing saccharide for the at least one cell to produce the biological product; and
optionally purifying the biological product from the fermentation broth.

In an embodiment of the use according to the third aspect and/or the method according to the fourth aspect, said living cell is a prokaryotic cell or a eukaryotic dell. Appropriate cells include yeast, bacteria, archaebacteria, fungi, insect cells, plant cells and animal cells, including mammalian cells (such as human cells and cell lines).

In an additional and/or alternative embodiment, the prokaryotic cell is a bacterial cell, preferably selected from the genus selected from the group consisting of Bacillus, Lactobacillus, Lactococcus, Enterococcus, Bifidobacterium, Sporolactobacillus spp., Micromomospora spp., Micrococcus spp., Rhodococcus spp., and Pseudomonas. Suitable bacterial species are Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, Bacillus circulans, Bifidobacterium longum, Bifidobacterium infantis, Bifidobacterium bifidum, Citrobacter freundii, Clostridium cellulolyticum, Clostridium ljungdahlii, Clostridium autoethanogenum, Clostridium acetobutylicum, Corynebacterium glutamicum, Enterococcus faecium, Enterococcus thermophiles, Escherichia coli, Erwinia herbicola (Pantoea agglomerans), Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, Lactococcus lactis, Pantoea citrea, Pectobacterium carotovorum, Proprionibacterium freudenreichii, Pseudomonas fluorescens, Pseudomonas aeruginosa, Streptococcus thermophiles and Xanthomonas campestris.

In an additional and/or alternative embodiment, the eukaryotic cell is a yeast cell, an insect cell, a plant cell or a mammalian cell. The yeast cell is preferably selected from the group consisting of Saccharomyces sp., in particular Saccharomyces cerevisiae, Saccharomycopsis sp., Pichia sp., in particular Pichia pastoris, Hansenula sp., Kluyveromyces sp., Yarrowia sp., Rhodotorula sp., and Schizosaccharomyces sp.

In an embodiment of the use according to the third aspect and/or the method according to the fourth aspect, said biological product is a human milk oligosaccharide. The human milk oligosaccharide may be selected from the group consisting of 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-neofucopentaose V, lacto-N-difucohexaose I, lacto-N-difucosylhexaose II, para-Lacto-N-fucosylhexaose, fucosyl-lacto-N-sialylpentaose b, fucosyl-lacto-N-sialylpentaose c, fucosyl-lacto-N-sialylpentaose c, disialyl-lacto-N-fucopentaose, 3-fucosyl-3′-sialyllactose, 3-fucosyl-6′-sialyllactose, lacto-N-neodifucohexaose I, 3′-sialyllactose, 6′-sialyllactose, sialyllacto-N-tetraoses LST-a, LST-b, LST-c, and disialyllacto-N-tetraose.

In an additional and/or alternative embodiment of the use according to the third aspect and/or the method according to the fourth aspect, said reducing saccharide is lactose. This embodiment is of particular advantage wherein lactose has to be supplied to the fermentation broth for the living cells for producing the desired human milk oligosaccharide. Being able to reduce or avoid the formation of lactulose upon heat sterilization of an aqueous lactose solution by the method according to the first aspect, eliminates the need of removing lactulose from the HMO preparation when the HMO preparation shall be used for manufacturing a nutritional formula, especially an infant formula, a medicinal food or a dietary supplement.

Using a thermally treated aqueous saccharide solution containing at least one reducing saccharide, wherein the aqueous saccharide solution has been acidified as described herein before prior to and/or in the course of its thermal treatment in a fermentative production process provides additional advantages. First, the present invention permits sterilization of an aqueous solution containing a reducing saccharide by heat sterilization methods without or with reduced isomerization of the reducing saccharide. Hence, aqueous solutions containing a reducing saccharide do not have to be sterilized by sterile filtration, which is less reliable than heat sterilization (e.g. with regard to elimination of bacteriophages), in particular if large volumes of an aqueous saccharide solution, such as several cubic meters, need to be sterilized. In addition, heat sterilization of large volumes is more economic than sterile filtration. Second, the present invention permits providing aqueous saccharide solutions containing at least one reducing saccharide, wherein the concentrations of the at least one reducing saccharide is higher than the saturation concentration of the at least one reducing saccharide at room temperature, as the aqueous saccharide solution can be heated and kept at an elevated temperature, i.e. a temperature above room temperature. Moreover, keeping an aqueous saccharide solution at an elevated temperature reduces the viscosity of the aqueous saccharide solution which in turn eases handling of the aqueous saccharide solution, for example when pumping the aqueous saccharide solution through a pipe or a hose. Third, being able to provide an aqueous saccharide solution having an increased saccharide concentration permits obtaining higher product yields in a fermentative production process. This is because the volume of a fermenter is limited and the volume of the fermentation broth in a fermenter increases during a fermentation process due to the supply of—among others—an aqueous saccharide solution to the fermentation broth which aqueous saccharide solution is required for the production of the desired biological product by the cells being cultivated. The higher the concentration of the saccharide in the aqueous saccharide solution the smaller the volume of the aqueous saccharide solution that needs to be added to a fermentation broth in order to obtain and/or maintain a predetermined concentration of the at least one reducing saccharide in the fermentation broth. Thus, using an aqueous saccharide solution according to the invention having an increased saccharide concentration, a higher saccharide concentration in the fermentation broth in a given fermenter can be achieved or a predetermined saccharide concentration can be maintained for a longer time period as the volume in the fermenter being available for supplies is depleted more slowly. This in turn provides more of the reducing saccharide to the cells for producing the desired biological product, and hence increases the yield of the desired biological product that can be obtained in a single batch fermentation. Therefore, the desired biological product can be produced—at the end of the fermentation process—in an amount of 100 g/L in the fermentation broth, preferably in an amount of ≥150 g/L in the fermentation broth, more preferably in an amount of ≥200 g/L in the fermentation broth. For example amounts of 2′-fucosyllactose of more than 100 g/L, namely of about 150 g/L were obtained when a acidified 0.66 M aqueous lactose solution was used which was heat sterilized, and even higher yields can be obtained if the lactose concentration in the aqueous solution to be fed to the fermentation broth is further increased.

In another embodiment of the use according to the third aspect and/or the method according to the fourth aspect, the at least one reducing saccharide is lactose, and said biological product is selected from the group consisting of lactosucrose and lactobionic acid.

Thus, according to the fifth aspect, the invention provides a biological product which has been produced by one of the methods according to the fourth aspect.

In an embodiment, the biological product is a human milk oligosaccharide, preferably a human milk oligosaccharide selected from the group consisting of 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-neofucopentaose V, lacto-N-difucohexaose I, lacto-N-difucosylhexaose II, para-Lacto-N-fucosylhexaose, fucosyl-lacto-N-sialylpentaose b, fucosyl-lacto-N-sialylpentaose c, fucosyl-lacto-N-sialylpentaose c, disialyl-lacto-N-fucopentaose, 3-fucosyl-3′-sialyllactose, 3-fucosyl-6′-sialyllactose, lacto-N-neodifucohexaose I, 3′-sialyllactose, 6′-sialyllactose, sialyllacto-N-tetraoses LST-a, LST-b, LST-c, and disialyllacto-N-tetraose.

In an alternative embodiment, the biological product is selected from the group consisting of lactosucrose and lactobionic acid or derivatives of the above mentioned human milk oligosaccharides.

According to a further aspect, the invention provides the use of the biological product for manufacturing a formulation.

In an embodiment of the use of the biological product for manufacturing a formulation, the biological product is a human milk oligosaccharide, selected from the group consisting of 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-neofucopentaose V, lacto-N-difucohexaose I, lacto-N-difucosylhexaose II, para-Lacto-N-fucosylhexaose, fucosyl-lacto-N-sialylpentaose b, fucosyl-lacto-N-sialylpentaose c, fucosyl-lacto-N-sialylpentaose c, disialyl-lacto-N-fucopentaose, 3-fucosyl-3′-sialyllactose, 3-fucosyl-6′-sialyllactose, lacto-N-neodifucohexaose I, 3′-sialyllactose, 6′-sialyllactose, sialyllacto-N-tetraoses LST-a, LST-b, LST-c, and disialyllacto-N-tetraose. The formulation in this embodiment is selected from the group consisting of nutritional formulations, preferably infant formula, medicinal food and dietary supplements.

Provided that the reducing saccharide is lactose, the method of inhibiting isomerization according to the first aspect may provide a heat sterilized lactose solution without or with reduced amounts of lactulose for fermentative production of a human milk oligosaccharide. Said human milk oligosaccharide may then be employed in the manufacturing of a nutritional formulation, preferably an infant formula, which does not contain or contains less amount of epilactose, lactulose and/or a derivative of lactulose such as fucosyllactulose (without the need of removing lactulose or its derivative from the HMO preparation).

According to a further aspect, provided are formulations comprising at least one biological product that has been produced by a biotechnological production process as described herein before. Said formulation is preferably selected from the group consisting of nutritional formulations, preferably infant formula, medicinal food and dietary supplements.

The present invention will be described with respect to particular embodiments and with reference to drawings, but the invention is not limited thereto but only by the claims. Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described to herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description and drawings provided herein, numerous specific details are set forth. However, it is understood that embodiments of the invention may be practiced without these specific details. In other instances, well-known methods, structures and techniques have not been shown in detail in order not to obscure an understanding of this description.

The invention will now be described by a detailed description of several embodiments of the invention. It is clear that other embodiments of the invention can be configured according to the knowledge of persons skilled in the art without departing from the true spirit or technical teaching of the invention, the invention being limited only by the terms of the appended claims.

### Example 1—Acidification of Lactose with Organic Acids Prior to Heat Sterilization

A 0.66 M lactose solution was prepared by dissolving 226 g of lactose in water. The final volume of the solution was 1 litre. At a temperature of 30° C. to 35° C. the pH was adjusted by using 50% (w/v) citrate or 99% (v/v) acetic acid. Afterwards, the solution was sterilized in a vertical autoclave (Systec VX-65, Linden, Germany) at 121° C. for 20 minutes. Samples were taken before and after heat sterilization and kept frozen prior to analysis by high performance liquid chromatography (HPLC). HPLC was carried out using a RID-10A refractive index detector (Shimadzu, Germany) and a Waters XBridge Amide Column 3.5 μm (250×4.6 mm) (Eschborn, Germany) connected to a Shimadzu HPLC system. Isocratic elution was carried out with 30% solvent A (50% (v/v) acetonitrile in double distilled water, 0.1% (v/v) NH4OH) and 70% solvent B (80% (v/v) acetonitrile in double distilled water, 0.1% (v/v) NH4OH) at 35° C. and at a flow rate of 1.4 mL min-1. Samples were cleared by solid phase extraction on an ion exchange matrix (Strata ABW, Phenomenex). Ten microliter of the sample (dilution of 1:5) was applied to the column. Finally, the relative amount of detected sugars was determined. As depicted in tables 1 and 2, the heat sterilization induced lactose isomerization decreased with decreasing pH values of the solutions prior to heat treatment. No lactulose formation could be observed at pH 4.0 to 3.0 or pH 3.0 when acidification was carried out with citrate or acetic acid, respectively. The acid-catalysed degradation of lactose into its monosaccharides was increased with lower pH values but was not detectable at pH 4.5.

TABLE 1
Relative amount of sugars detected in pH adjusted 0.66M lactose
solutions before and after heat sterilization. The pH adjustment
was carried out using 50% (w/v) citrate. Depicted is the percental
amount of sugars (area under the curve; AUC) detected by HPLC.

| | Relative composition [%] | | |
|---|---|---|---|
| pH | Monosaccharides | Lactulose | Lactose |

before heat sterilization

| | | | |
|---|---|---|---|
| 3.0-6.8 | n.d. | n.d. | 100 |

after heat sterilization

| | | | |
|---|---|---|---|
| 6.8 (no pH | 0.44 | 6.16 | 93.40 |
| adjustment) | | | |
| 5.0 | 0.27 | 0.87 | 98.85 |
| 4.5 | n.d. | 0.15 | 99.85 |
| 4.0 | 0.68 | n.d. | 99.32 |
| 3.5 | 1.87 | n.d. | 98.13 |
| 3.0 | 5.96 | n.d. | 94.04 |

TABLE 2
Relative amount of sugars detected in pH adjusted 0.66M
lactose solutions before and after heat sterilization.
The pH adjustment was carried out using 80% (v/v) acetic
acid. Depicted is the percental amount of sugars (area
under the curve; AUC) detected by HPLC.

| | Relative composition [%] | | |
|---|---|---|---|
| pH | Monosaccharides | Lactulose | Lactose |

before heat sterilization

| | | | |
|---|---|---|---|
| 3.0-6.8 | n.d. | n.d. | 100 |

after heat sterilization

| | | | |
|---|---|---|---|
| 6.8 (no pH | 0.44 | 6.16 | 93.40 |
| adjustment) | | | |
| 5.0 | 0.21 | 1.13 | 98.65 |
| 4.5 | n.d. | 0.31 | 99.69 |
| 4.0 | 0.49 | 0.12 | 99.39 |
| 3.5 | 1.41 | 0.07 | 98.52 |
| 3.0 | 4.35 | n.d. | 95.65 |

### Example 2—Acidification of Lactose with Inorganic Acids Prior to Heat Sterilization

A 0.66 M lactose solution was prepared by dissolving 226 g of lactose in water. The final volume of the solution was 1 litre. At a temperature of 30° C. to 35° C. the pH was adjusted by using 37% (v/v) hydrochloric acid, 50% (v/v) phosphoric acid or 96 (v/v) sulfuric acid. Afterwards, the solution was sterilized in a vertical autoclave (Systec VX-65, Linden, Germany) at 121° C. for 20 minutes. Samples were taken before and after heat sterilization and kept frozen prior to analysis by high performance liquid chromatography (HPLC). HPLC was carried out using a RID-10A refractive index detector (Shimadzu, Germany) and a Waters XBridge Amide Column 3.5 μm (250×4.6 mm) (Eschborn, Germany) connected to a Shimadzu HPLC system. Isocratic elution was carried out with 30% solvent A (50% (v/v) acetonitrile in double distilled water, 0.1% (v/v) NH4OH) and 70% solvent B (80% (v/v) acetonitrile in double distilled water, 0.1% (v/v) NH4OH) at 35° C. and at a flow rate of 1.4 mL min-1. Samples were cleared by solid phase extraction on an ion exchange matrix (Strata ABW, Phenomenex). Ten microliter of the sample (dilution of 1:5) was applied to the column. Finally, the relative amount of detected sugars was determined. As depicted in tables 3, 4 and 5, the heat sterilization induced lactose isomerization decreased in consequence of increased acidification of the solutions prior to heat treatment. No lactulose formation could be observed at pH 3.5 to 3.0. Contrarily, the acid-catalysed degradation of lactose into its monosaccharides was increased with lower pH values but was absent at pH values 4.5 to 4.0 or pH 5.0 to 4.0 when phosphoric acid and sulfuric acid or hydrochloric acid was used for acidification, respectively.

TABLE 3
Relative amount of sugars detected in pH adjusted 0.66M
lactose solutions before and after heat sterilization.
The pH adjustment was carried out using 37% (v/v) hydrochloric
acid. Depicted is the percental amount of sugars (area
under the curve; AUC) detected by HPLC.

| | Relative composition [%] | | |
|---|---|---|---|
| pH | Monosaccharides | Lactulose | Lactose |

before heat sterilization

| | | | |
|---|---|---|---|
| 3.0-6.8 | n.d. | n.d. | 100 |

after heat sterilization

| | | | |
|---|---|---|---|
| 6.8 (no pH | 0.44 | 6.16 | 93.40 |
| adjustment) | | | |
| 5.0 | n.d. | 0.57 | 99.43 |
| 4.5 | n.d. | 0.17 | 99.83 |
| 4.0 | n.d. | 0.20 | 99.80 |
| 3.5 | 1.50 | n.d. | 98.50 |
| 3.0 | 5.62 | n.d. | 94.38 |

TABLE 4
Relative amount of sugars detected in pH adjusted 0.66M
lactose solutions before and after heat sterilization.
The pH adjustment was carried out using 50% (v/v) phosphoric
acid. Depicted is the percental amount of sugars (area
under the curve; AUC) detected by HPLC.

| | Relative composition [%] | | |
|---|---|---|---|
| pH | Monosaccharides | Lactulose | Lactose |

before heat sterilization

| | | | |
|---|---|---|---|
| 3.0-6.8 | n.d. | n.d. | 100 |

after heat sterilization

| | | | |
|---|---|---|---|
| 6.8 (no pH | 0.44 | 6.16 | 93.40 |
| adjustment) | | | |
| 5.0 | 0.30 | 0.49 | 99.21 |
| 4.5 | n.d. | 0.10 | 99.90 |
| 4.0 | n.d. | 0.08 | 99.92 |
| 3.5 | 1.40 | n.d. | 98.60 |
| 3.0 | 4.44 | n.d. | 95.56 |

TABLE 5
Relative amount of sugars detected in pH adjusted 0.66M
lactose solutions before and after heat sterilization.
The pH adjustment was carried out using 96% (v/v) sulfuric
acid. Depicted is the percental amount of sugars (area
under the curve; AUC) detected by HPLC.

| | Relative composition [%] | | |
|---|---|---|---|
| pH | Monosaccharides | Lactulose | Lactose |

before heat sterilization

| | | | |
|---|---|---|---|
| 3.0-6.8 | n.d. | n.d. | 100 |

after heat sterilization

| | | | |
|---|---|---|---|
| 6.8 (no pH | 0.43 | 6.34 | 93.24 |
| adjustment) | | | |
| 5.0 | 0.26 | 0.67 | 99.05 |
| 4.5 | n.d. | 0.17 | 99.83 |
| 4.0 | n.d. | 0.17 | 99.82 |
| 3.5 | 1.11 | n.d. | 98.89 |
| 3.0 | 3.21 | n.d. | 96.79 |

### Example 3—Improved Production Process of 2′-Fucosyllactose

An engineered E. coli BL21 (DE3) ΔnagAb ΔwcaJ ΔfuclK ΔpfkA strain was used in accordance with European patent application 16 196 486, overexpressing enzymes for de novo synthesis of GDP-Fucose (ManB, ManC, Gmd, WcaG), the bifunctional L-fucokinase/L-fucose 1-phosphat guanylyltranferase of Bacteroides fragilis, the 2-fucosyltransferase gene wbgL from E. coli:O126, the lactose permease gene lacy, the sugar efflux transporter yberc0001_9420 from Yersinia bercovieri ATCC 43970, the fructose-1,6-bisphosphate aldolase (fbaB) and a heterologous fructose-1,6-bisphosphate phosphatase (fbpase) from Pisum sativum.

The E. coli strain was cultivated in a 3 L fermenter at 33° C. in a mineral salts medium that contains 3 g/L KH₂PO₄, 12 g/L K₂HPO₄, 5 g/L (NH₄)₂SO₄, 0.3 g/L citric acid, 2 g/L MgSO₄×7H₂O, 0.1 g/L NaCl and 0.015 g/L CaCl₂×6H₂O with 1 mL/L trace element solution (54.4 g/L ammonium ferric citrate, 9.8 g/L MnCl₂×4H₂O, 1.6 g/L CoCl₂×6H₂O, 1 g/L CuCl₂×2H₂O, 1.9 g/L H₃BO₃, 9 g/L ZnSO₄×7H₂O, 1.1 g/L Na₂MoO₄×2H₂O, 1.5 g/L Na₂SeO₃, 1.5 g/L NiSO₄×6H₂O), 2% (v/v) glycerol as Lo sole carbon- and energy source as well as 60 mM of heat sterilized lactose, which was acidified to pH 3.0 with 96% (v/v) sulfuric acid prior to sterilization. The pH was hold at 7.0 by titrating 25% ammonia. The fermenter was inoculated to an OD₆₀₀ of 0.1 with a pre-culture grown in the described medium but lacking lactose. After leaving the batch phase, indicated by a rise in the dissolved oxygen level, the glycerol feed (60% v/v) as well as the 0.66 M lactose feed (acidified to pH 3.0 using 96% (v/v) sulfuric acid prior to heat sterilization) was started. A concentration of 10-40 mM lactose was held throughout the production phase of the fermentation process, regulated according to HPLC-analyses. Glycerol (60% v/v) was fed with flow rates of 6-8 ml/L/h (referring to the starting volume). The fermentation was stopped when the filling volume in the tank reached its maximum. At this point, a 2′-fucosyllactose titer of 146 g/L was determined in the culture supernatant of the broth.

In course of 2′-fucosyllacose fermentation processes using sterile-filtered lactose, instead of acidified, heat sterilized lactose, comparable 2′-fucosyllactose titers were achieved. Furthermore, the kind and amounts of by-products detected in the culture broth of fermentations carried out with sterile-filtered or heat-sterilized (acidified) lactose were comparable. None of the by-products of the kind lactulose, epilactose, fucosyllactulose or fucosylepilactose as well as no other by-products which may originate from the addition of heat-sterilized lactose, were detected in the broth when acidified, heat-sterilized lactose was provided for the fermentation.

1. A method of inhibiting isomerization of a reducing saccharide in an aqueous solution comprising said reducing saccharide upon a thermal treatment of said aqueous saccharide solution, the method comprising acidifying the aqueous saccharide solution prior to and/or in the course of the thermal treatment.

2. The method according to claim 1, wherein the aqueous saccharide solution is acidified to a pH having a value of between about 1 to about 6, optionally to a pH having a value of between about 2 to about 5, and optionally to a pH having a value of between about 3 to about 4.

3. The method according to claim 1, wherein the aqueous saccharide solution is acidified by adding at least one acid to said aqueous saccharide solution.

4. The method according to claim 3, wherein the acid is an inorganic acid, optionally selected from the group consisting of hydrochloric acid, sulfuric acid, sulfurous acid, phosphoric acid, boric acid, hydrofluoric acid, hydrobromic acid, perchloric acid, hydroiodic acid and carbonic acid.

5. The method according to claim 3, wherein the acid is an organic acid, optionally selected from the group consisting of monocarboxylic acids, dicarboxylic acids, and tricarboxylic acids.

6. The method according to claim 1, wherein the reducing sugar is selected from the group consisting of aldoses, disaccharides and oligosaccharides, optionally a disaccharide selected from the group consisting of lactose and maltose.

7. The method according to claim 1, wherein the thermal treatment comprises exposing the acidified aqueous solution to a temperature in the range of about 30° C. to about 150° C.

8. A thermally treated aqueous solution comprising at least one reducing saccharide, wherein the aqueous solution is obtained by a method according to claim 1.

9. The thermally treated aqueous solution according to claim 8, wherein the aqueous solution is sterile.

10. The thermally treated aqueous solution according to claim 8, wherein the reducing saccharide is lactose, optionally present in a concentration of ≥10 mM, optionally in a concentration of ≥100 mM, optionally in a concentration of ≥0.66 M, and optionally in a concentration of ≥1 M.

11. A product comprising a thermally treated aqueous solution comprising at least one reducing saccharide according to claim 8 adapted for a biotechnological production process for producing a biological product.

12. The product according to claim 11, wherein the biotechnological process is a selected from the group consisting of a biocatalytic production process and a fermentative production process.

13. The product according to claim 12 for a fermentative production of a human milk oligosaccharide, wherein the thermally treated aqueous solution comprising at least one reducing saccharide is a wet heat sterilized aqueous solution comprising lactose.

14. A method of biotechnological production of a biological product, the method comprising:
providing at least one cell that is capable of producing the biological product;
cultivating the at least one cell in a fermentation broth containing and/or being supplemented with the thermally treated aqueous solution according to claim 8 for the at least one cell to produce the biological product; and
optionally purifying the biological product from the fermentation broth.

15. The method according to claim 14, wherein the biological product is a human milk oligosaccharide, and wherein the thermally treated aqueous solution is a wet heat sterilized aqueous solution comprising lactose.

16. The method according to claim 14, wherein the biological product is produced in an amount of ≥100 g/L in the fermentation broth, optionally in an amount of ≥150 g/L in the fermentation broth, optionally in an amount of ≥200 g/L in the fermentation broth at an end of the fermentation process.

17. A biological product manufactured by a process according to claim 14, wherein the biological product is optionally selected from the group consisting of a human milk oligosaccharide, lactosucrose and lactobionic acid.

18. The method according to claim 14, wherein the biological product is a human milk oligosaccharide being selected from the group consisting of 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-neofucopentaose V, lacto-N-difucohexaose I, lacto-N-difucosylhexaose II, para-Lacto-N-fucosylhexaose, fucosyl-lacto-N-sialylpentaose b, fucosyl-lacto-N-sialylpentaose c, fucosyl-lacto-N-sialylpentaose c, disialyl-lacto-N-fucopentaose, 3-fucosyl-3′-sialyllactose, 3-fucosyl-6′-sialyllactose, lacto-N-neodifucohexaose I, 3′-sialyllactose, 6′-sialyllactose, sialyllacto-N-tetraoses LST-a, LST-b, LST-c, and disialyllacto-N-tetraose.
